(19) United States
(12) Patent Application Publication
(10) Pub. No.: US 2023/0373921 A1
(43) Pub. Date: Nov. 23, 2023

(54) Nicotinic Acid Derivative B Having Anti-Inflammatory and Immunosuppressive Activity and Use Thereof

(71) Applicant: Chongqing Academy of Chinese Materia Medica, Chongqing (CN)

(72) Inventors: Yanlei GUO, Chongqing (CN); Yong YANG, Chongqing (CN); Dajian YANG, Chongqing (CN)

(21) Appl. No.: 18/029,302

(22) PCT Filed: Sep. 29, 2020
(86) PCT No.: PCT/CN2020/118819

§ 371 Date: Mar. 29, 2023

(51) Int. Cl. C07D 213/80; A61P 29/00

(52) U.S. Cl. CPC C07D 213/80; A61P 29/00

(57) ABSTRACT

Provided in the present invention is a nicotinic acid derivative B having an anti-inflammatory and immunosuppressive activity, and the structural general formula thereof is I, wherein, R₁ and R₂ are different substitution sites on a main chain, and R₁₁′, R₂₁′, R₃₁′, R₁₂′, R₂₂′, and R₃₂′ are different substitution sites on a side chain. The nicotinic acid derivative B compound provided in the present invention has a good anti-inflammatory and anti-autoimmune disease activity, and a high selectivity.

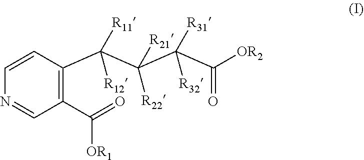

### TECHNICAL FIELD

The present disclosure relates to a compound, and more particularly, to a nicotinic acid derivative B having anti-inflammatory and immunosuppressive activity separated from medicinal materials of genus Tripterygium and use thereof.

### BACKGROUND

During long-term clinical pattern identification as the basis for determining treatment, traditional Chinese medicine (TCM) has a long history of Tripterygium plants of the family Celastraceae in the treatment of wind-dampness arthromyodynia. With a cold nature, a bitter taste and toxicity, Tripterygium plants have the effects of dispelling wind and eliminating dampness, soothing the sinews and activating collaterals, clearing away the heat-evil and detoxicating. As clinically important immunosuppressants, Tripterygium plants play an important role in the treatment of autoimmune diseases including rheumatoid arthritis (RA), primary nephrotic syndrome and systemic lupus erythematosus.

Tripterygium plants mainly include Tripterygium hypoglaucum (Levl.) Hutch, Tripterygium regelii Sprague et Takeda, and Tripterygium wilfordii Hook. f. According to the statistical data on the official website of the National Medical Products Administration, 45 preparations related to medicinal materials of genus Tripterygium from 43 drug manufacturing enterprises in China are listed on the market at present. The chemical composition of Tripterygium plants mainly includes sesquiterpene alkaloids, diterpenes, and triterpenes. The total alkaloid content of T. wilfordii Hook. f. is about 0.07-0.29%, and such components play an important role in immunosuppression. The sesquiterpene alkaloids are structurally characterized by a dihydrofuran-type sesquiterpene, and the reported structure mainly comprises three types, namely, wilfordate/evoniate, hydroxy-wilfordate/evoniate, and iso-wilfordate/evoniate, including a total of 71 sesquiterpene alkaloids. Sesquiterpene alkaloids from T wilfordii Hook. f. are a group of sesquiterpene compounds having high oxygen content, and the structure is characterized by containing a special macrodiolide skeleton, comprising two parts: 2-(carboxyalkyl) nicotinic acid and polyoxygenated dihydro-beita-agarofuran sesquiterpenoid. The hydroxyl groups of sesquiterpene moieties are generally esterified by various organic acids, including acetic acid, benzoic acid, furanic acid, nicotinic acid, and fatty acids; the 2-(carboxyalkyl) nicotinic acid moiety is mainly derived from acetylenic acid, vitamin acids, hydroxyvitamin acids, or homologs thereof, while the difference in the 2-(carboxyalkyl) nicotinic acid moiety is the core of the macrodiolide skeleton that is different, as well as the core of the structural diversity of the chemical composition of alkaloids from T wilfordii Hook. f.

In TCM, it has been found in long-term clinical pattern identification as the basis for determining treatment that Tripterygium plants of the family Celastraceae has exact efficacy in the treatment of wind-dampness arthromyodynia. Tripterygium plants have the effects of dispelling wind and eliminating dampness, soothing the sinews and activating collaterals, clearing away the heat-evil and detoxicating. Tripterygium plants play an important role in the treatment of autoimmune diseases including rheumatoid arthritis. The mechanism of medicinal materials of the genus Tripterygium in the treatment of rheumatoid arthritis is closely related to the key targets of the pathogenesis of inflammation and the immune regulation of T cells. One week after treating rheumatoid arthritis with Radix Tripterygii Hypoglauci, it has obvious inhibitory effects on the secondary foot swelling degree and the arthritis index of the adjuvant arthritis model rat; it obviously reduces the concentrations of proinflammatory factors IL-1α and IL-1β and functional protein MMP3 in serum, and obviously raises the concentrations of anti-inflammatory factors IL-4 and IL-10 in serum; three weeks after administration, the proportion of regulatory T cells (Tregs) to T lymphocytes is obviously higher than that of the model group, and the therapeutic effect is obviously shown. The applicants conduct deep and systematic research on immunosuppressive active ingredients in medicinal materials of the genus Tripterygium, and the research has successively funded by National and Provincial Scientific Research Projects (more than RMB 15 million). Related subjects include: Major National R&D Project of Major New Drug Development of the Ministry of Science and Technology of China: New Drug Research on Haoteng Qufeng Capsules in the Treatment of Rheumatoid Arthritis (Project Number: 2017ZX09101002-002-004); project of the Chongqing Municipal Health Bureau: “Research on the Large Variety Technology Improvement and Indication Expansion of Huobahuagen Tablets (Project Number: cstc2013jcsf10011)”; project of the Chongqing Municipal Science and Technology Bureau: “Large Variety Industrial Promotion and Improvement of Huobahuagen Tablets” (Project Number: cstc2014jcsf10001); project of the Chongqing Municipal Science and Technology Bureau: Research on the Mechanism of the Immunosuppression of Huobahuagen Tablets in Rheumatoid Arthritis Based on Metabonomics of TCM (Project Number: 2015cstc-jbky-01913); and project of the Chongqing Municipal Science and Technology Bureau: Pharmacokinetic Research on Wilforine in Beagles (Project Number: cstc2018jxj1130055).

### SUMMARY

On the basis of the above-mentioned prior art, the present disclosure provides a nicotinic acid derivative B compound having anti-inflammatory activity, and a general molecular formula thereof is:

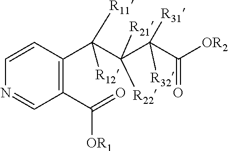

where R₁ and R₂ are different substitution sites on a main chain, and R₁₁′, R₂₁′, R₃₁′, R₁₂′, R₂₂′, and R₃₂′ are different substitution sites on a side chain.

The structural formula is as follows:

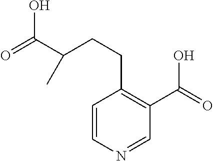 [SMILES: CC(CCc1ccncc1C(=O)O)C(=O)O]

The structural formula is as follows:

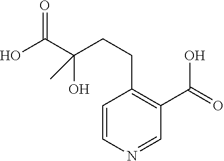 [SMILES: CC(O)(CCc1ccncc1C(=O)O)C(=O)O]

The structural formula is as follows:

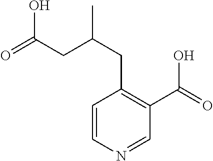 [SMILES: CC(CC(=O)O)Cc1ccncc1C(=O)O]

Use of a nicotinic acid derivative B having immunosuppressive activity in the preparation of a medicament for the treatment of rheumatoid arthritis, psoriasis, and other autoimmune diseases is provided.

Use of a nicotinic acid derivative B with anti-inflammatory activity in the preparation of an anti-inflammatory medicament is provided.

Use of a nicotinic acid derivative B having anti-platelet aggregation activity in the preparation of an anti-platelet aggregation medicament is provided.

The present disclosure has the following beneficial technical effects: The nicotinic acid derivative B compound provided in the present disclosure has excellent anti-inflammatory and anti-autoimmune disease activity, strong selectivity, and excellent clinical application value.

### BRIEF DESCRIPTION OF THE DRAWINGS

FIG. 1 is an ESI-MS spectrum of a nicotinic acid derivative B-1;

FIG. 2 is a 1H-NMR spectrum of a nicotinic acid derivative B-1;

FIG. 3 is an ESI-MS spectrum of a nicotinic acid derivative B-2;

FIG. 4 is a 1H-NMR spectrum of a nicotinic acid derivative B-2;

FIG. 5 is an ESI-MS spectrum of a nicotinic acid derivative B-3;

FIG. 6 is a 1H-NMR spectrum of a nicotinic acid derivative B-3;

FIGS. 7A-D illustrate anti-inflammatory activity of a nicotinic acid derivative B;

FIGS. 8A-F illustrate anti-RA activity of a nicotinic acid derivative B;

FIG. 9 illustrates hypoglycemic activity of a nicotinic acid derivative B;

FIGS. 10A-B illustrate a docking result of a nicotinic acid derivative B and 3KVJ;

FIGS. 11A-B illustrate a docking result of a nicotinic acid derivative B and 5CJF;

FIGS. 12A-B illustrate a docking result of a nicotinic acid derivative B and 5LYW;

FIGS. 13A-B illustrate a docking result of a nicotinic acid derivative B and 6CSD; and

FIGS. 14A-B illustrate a docking result of a nicotinic acid derivative B and 6IIV.

### DETAILED DESCRIPTION OF THE EMBODIMENTS

### Example 1 Preparation Method of a Nicotinic Acid Derivative

Root barks of Radix Tripterygii Hypoglauci were pulverized; 1 kg of medicinal material powder was weighed, placed in a 5,000 mL round-bottom flask, and reflux-extracted with 3,000 mL and 2,000 mL of water, respectively; before each extraction, the medicinal material powder was soaked for 60 min, and reflux-extracted for 60 min, respectively; two extracts were combined and concentrated into a paste, and the extractum was diluted with a small volume of water and extracted with 1.5 times, 1 time and 0.5 times the volume of ethyl acetate, respectively. (1) After the ethyl acetate layers were combined, the organic phase was recovered; the extractum was evaporated to dryness for 5 g, dissolved in 7.5 mL of pure methanol, and reacted in a water bath with 2.5 mL of 0.4 mol/L NaOH in methanol at a constant temperature of 60° C. for 4 h; the reaction mixture was evaporated to dryness to remove methanol, the residues were diluted with 10 mL of water and extracted with 2 times the volume of ethyl acetate thrice, and the aqueous layer was evaporated to dryness. The aqueous layer was loaded on a column by the wet process and passed through a C18 column, and eluted with 5% methanol-water at a natural flow rate; eluents were collected in a segmented manner, and the eluent fractions were monitored by LC-MS. (2) The aqueous layer was concentrated to an extractum, and the extractum was dissolved in a small volume of water, and passed through a polyamide column. The column was eluted with 5 times the column volume of water; the eluent was collected, evaporated to dryness, redissolved with a small volume of water, stirred with 3 times the volume of 95% ethanol for 30 min, and centrifuged to collect a supernatant; the supernatant was evaporated to dryness, and dissolved with 5% methanol for liquid phase separation; after eluting with 15% methanol-water as a mobile phase, eluents were collected in a segmented manner, and eluent fractions were monitored by LC-MS.

### Example 2 Preparation Method of a Nicotinic Acid Derivative

Root barks of Radix Tripterygii Hypoglauci were pulverized; 1 kg of medicinal material powder was weighed, placed in a 5,000 mL round-bottom flask, and reflux-extracted with 3,000 mL and 2,000 mL of 80% ethanol solution, respectively; before each extraction, the medicinal material powder was soaked for 60 min, reflux-extracted for 60 min, respectively, and adjusted to pH 4.0; two extracts were combined, ethanol was recovered, and the extractum was diluted with a small volume of water and extracted with 1.5 times, 1 time and 0.5 times the volume of ethyl acetate, respectively. After the ethyl acetate layers were combined, the organic phase was recovered; the extractum was evaporated to dryness for 5 g, dissolved in 7.5 mL of pure methanol, and reacted in a water bath with 2.5 mL of 0.4 mol/L NaOH in methanol at a constant temperature of 60° C. for 4 h; the reaction mixture was evaporated to dryness to remove methanol, the residues were diluted with 10 mL of water and extracted with 2 times the volume of ethyl acetate thrice, and the aqueous layer was evaporated to dryness. The aqueous layer was loaded on a column by the wet process and passed through a C18 column, and eluted with 5% methanol-water at a natural flow rate; eluents were collected in a segmented manner, and the eluent fractions were monitored by LC-MS. The above monomer was obtained by preparing a liquid phase.

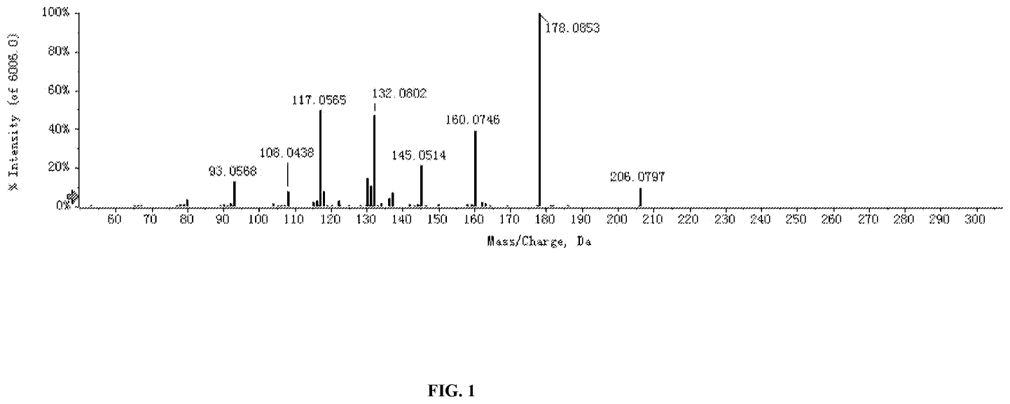

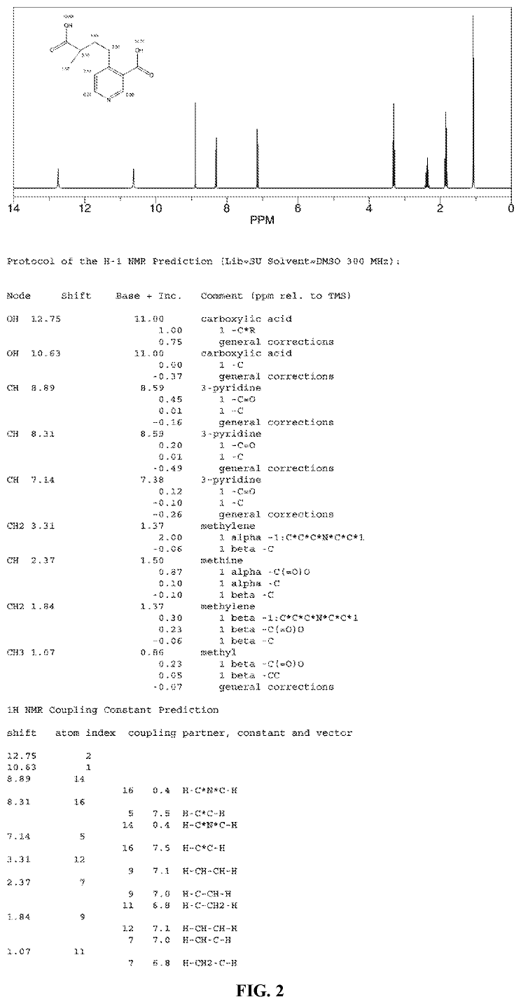

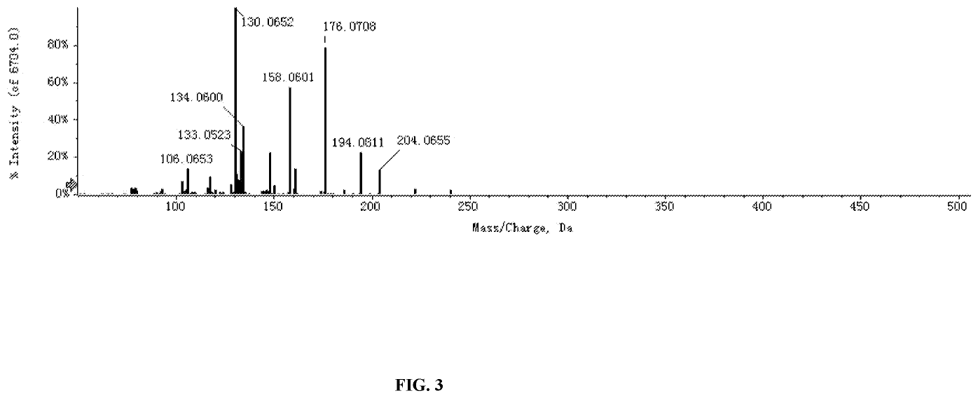

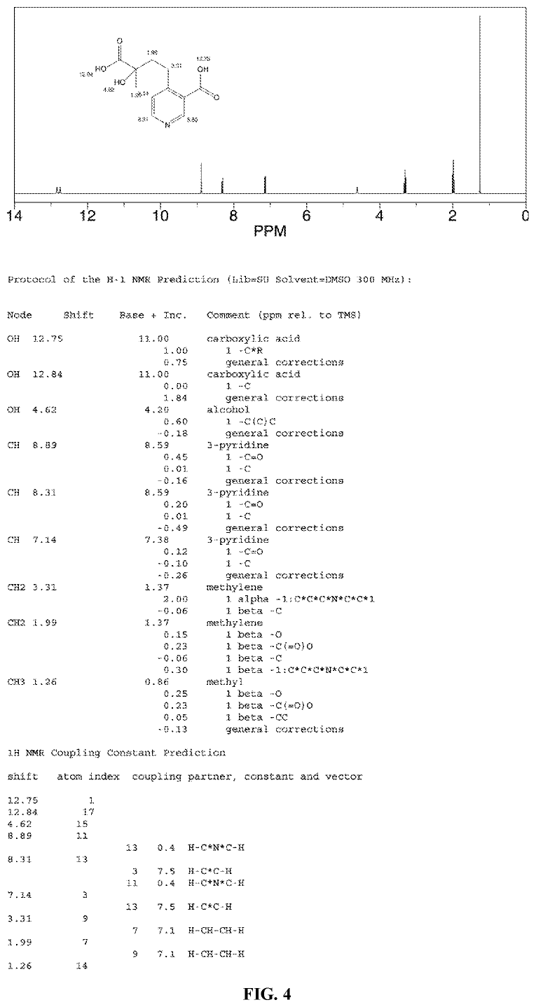

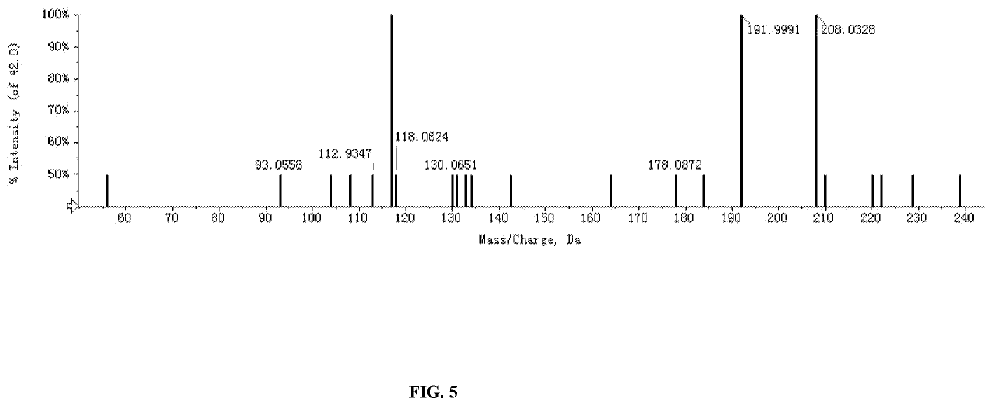

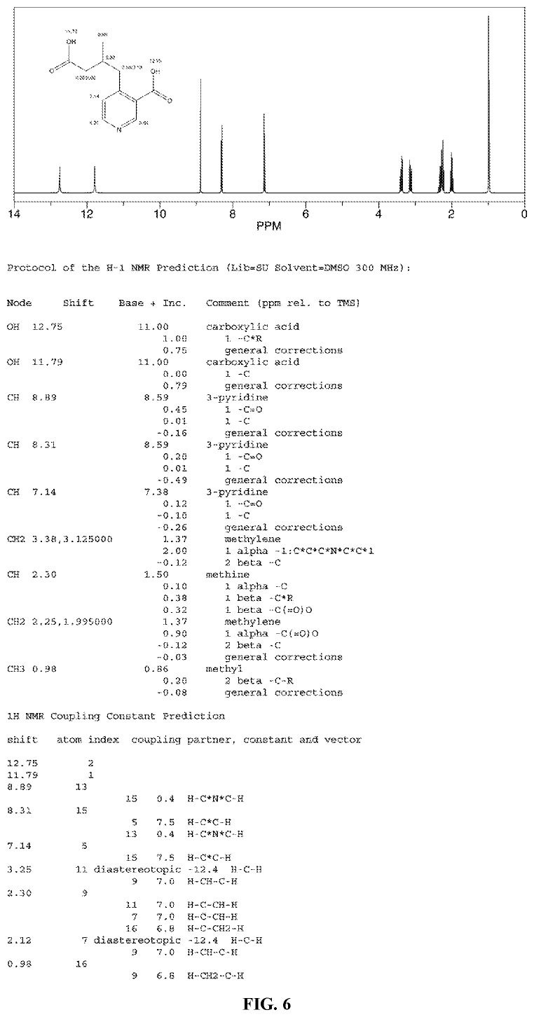

¹H-NMR chemical structure analysis and ESI-MS chemical structure analysis were conducted on the nicotinic acid derivative I compound obtained from the above synthesis, and the ¹H-NMR structure analysis results are shown in Table 1. The specific results are shown in FIGS. 1 to 6.

TABLE 1
¹H-NMR structure analysis of nicotinic acid derivative compound

| Name | ¹H-NMR: (TMS, D₂O, 500 MHz) |
|---|---|
| Nicotinic | ¹H NMR (500 MHz, D₂O) δ 8.89 (s, 1H), 8.31 (d, J = 7.5 |
| acid deriv- | Hz, 1H), 7.14 (d, J = 7.5 Hz, 1H), 3.31 (t, J = 7.1 Hz, 2H), |
| ative B-1 | 2.37 (q, J = 7.0 Hz, 6.8 Hz, 1H), 1.84 (q, J = 7.1 Hz, 7.0 |
| | Hz, 2H), 1.07 (d, J = 6.8 Hz, 3H). |
| Nicotinic | ¹H NMR (500 MHz, D₂O) δ 8.89 (s, 1H), 8.31 (d, J = 7.5 |
| acid deriva- | Hz, 1H), 7.14 (d, J = 7.5 Hz, 1H), 3.31 (t, J = 7.1 Hz, 2H), |
| tive B-2 | 1.99 (t, J = 7.1 Hz, 2H), 1.26 (d, J = 7.1 Hz, 3H). |
| Nicotinic | ¹H NMR (500 MHz, D₂O) δ 8.89 (s, 1H), 8.31 (d, J = 7.5 |
| acid deriv- | Hz, 1H), 7.14 (d, J = 7.5 Hz, 1H), 3.61 (d, J = 7.1 Hz, 2H), |
| ative B-3 | 2.30 (d, 2H), 1.30 (s, 3H), 0.98 (s, H)). |

ESI-MS chemical structure analysis was conducted on the nicotinic acid derivative obtained from the above synthesis, specifically as shown in Table 2:

TABLE 2
ESI-MS structure analysis of nicotinic acid derivative

| | Triple-Tof high resolution mass spectrometry |
|---|---|
| Name | (ESI_Positive) |
| Nicotinic | ESI-Positive (Triple-Tof 4600) |
| acid deriv- | [M + H]⁺: 224.0914 |
| ative B-1 | 206.0812, 178.0860, 150.0548, 124.0391, 106.0649, 80.0497 |
| Nicotinic | ESI-Positive (Triple-Tof 4600) |
| acid deriv- | [M + H]⁺: 240.0863 |
| ative B-2 | 222.0759, 204.0652, 194.0807, 176.0702, 158.0596, |
| | 134.0595, 130.0646, 117.0572, 106.0652, 77.0388 |
| Nicotinic | ESI-Positive (Triple-Tof 4600) |
| acid deriv- | [M + H]⁺: 240.0863 |
| ative B-3 | 222.0759, 204.0652, 194.0807, 176.0702, 158.0596, |
| | 134.0595, 130.0646, 117.0572, 106.0652, 77.0388 |

### Example 3 Investigation of Anti-Inflammatory Activity of Nicotinic Acid Derivative B

Passage culture: Macrophages were subcultured in a 37° C. and 5% CO₂ incubator containing Dulbecco's Modified Eagle Medium (DMEM) supplemented with 10% fetal bovine serum (FBS) and 1% double antibodies; about 1×10⁷ cells were collected, incubated with a cell lysis buffer (Tris, pH 7.4, 150 mmol/L NaCl, 1% NP-40, protease inhibitor cocktail) on an ice bath for 30 min, and centrifuged at 12,000 r/min for 20 min at 4° C., and the supernatant was collected and stored.

Drug intervention: Macrophages were subcultured in a 37° C. and 5% CO₂ incubator containing DMEM supplemented with 10% FBS and 1% double antibodies, and about 1×10⁷ cells were collected. Grouping: A control PBS group, an unknown molecule group, and an unknown molecule+PBS group were arranged. The final concentrations of the different unknown molecules obtained in Examples 2 were determined according to specific experiments.

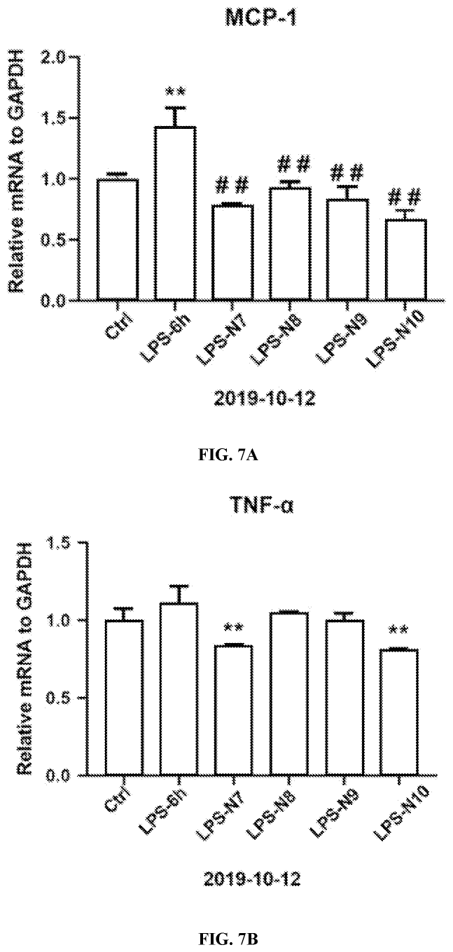

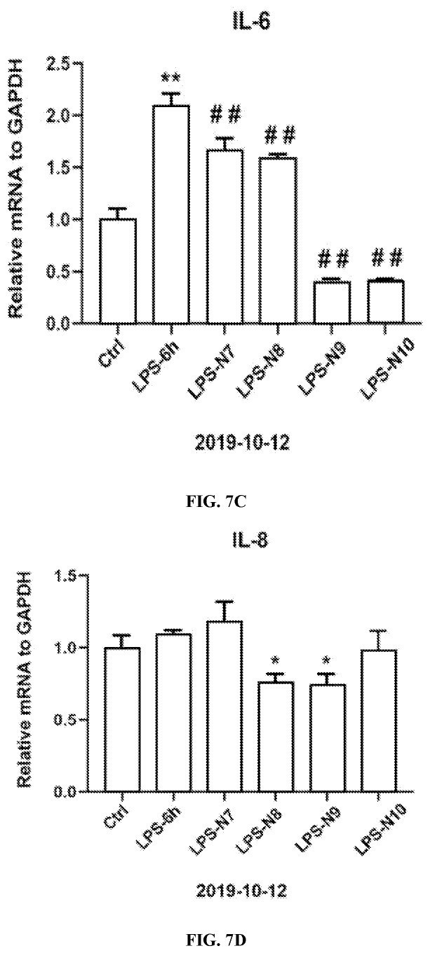

Based on the above-mentioned groups, after the macrophages were pretreated with different alkaloid molecules for 12 h, the following experiments were carried out: RT-PCR assay: to detect the transcription expression changes of inflammatory factors (IL-6, IL-8, MCP-1, and TNF-α) and a transcriptional regulator (PPARγ) in cells of each experimental group. The experimental results are shown in FIGS. 7A-D.

### Example 4 Investigation of Anti-RA Activity of Nicotinic Acid Derivative B

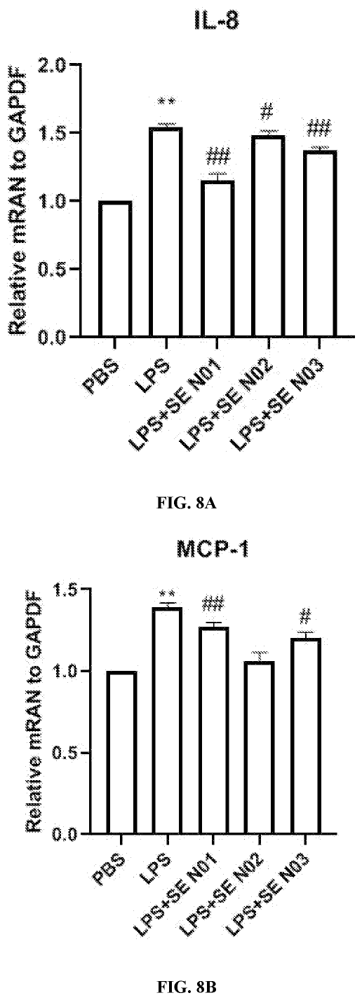

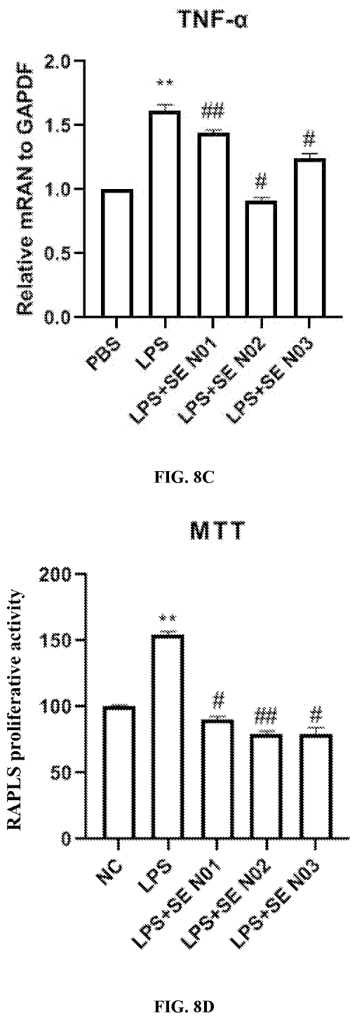

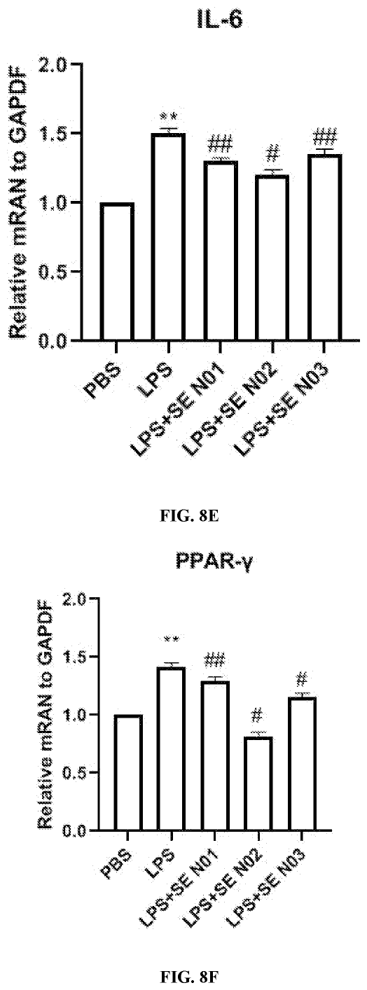

(1) A drug concentration-based toxicity test RA-FLSs (fibroblast-like synoviocytes) were cultured in a 37° C. and 5% CO₂ incubator containing DMEM supplemented with 10% FBS and 1% double antibodies, cells in logarithmic phase were collected, the concentration of cell suspension was adjusted, and the cell suspension was dispensed in a 96-well plate, 150 μL per well; after culturing for 24 h, 200 μL each of unknown samples of different concentrations was added to further culture until the desired time; the supernatant was discarded, 80 μL of fresh culture medium and 20 μL of MTT solution were added to further culture for 3 h; the supernatant was discarded, 100 μL of dimethyl sulfoxide (DMSO) was added into each well, the plate was shaken on a shaker at low speed for 10 min, the absorbance value of each well was measured at 570 nm, and the cell viability was calculated. (2) Detection of anti-RA activity The RA-FLSs were passaged and spread on a 6-well plate and divided into three groups, namely a control PBS group, an unknown molecule group, an unknown molecule+PBS group; the cells were cultured for 12 h after dosing, and cell RNA was extracted; after reverse transcription, RT-PCR assay was performed to detect the changes in the transcription expression levels of inflammatory factors (IL-6, IL-8, MCP-1, and TNF-α) and a transcriptional regulator (PPARγ) in cells of each experimental group. The experimental results are shown in FIGS. 8A-F.

### Example 5 Investigation of Hypoglycemic Activity of Nicotinic Acid Derivative B

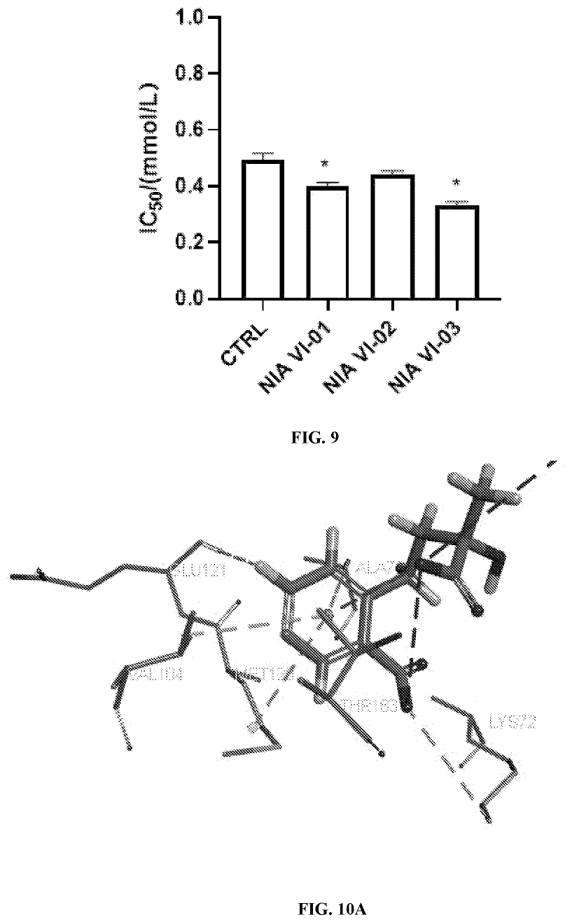

The inhibitory activity of the target compound tested against on platelet aggregation in rabbits induced by adenosine diphosphate (ADP) was detected by using Born's turbidimetry. The blood was drawn from the rabbit heart, anticoagulated with 3.8% (v/v) sodium citrate at a ratio of 1:9, and centrifuged at 1,000 r/min for 10 min to prepare platelet-rich plasma (PRP); the rest was partly centrifuged at 3,000 r/min for 15 min to prepare platelet-poor plasma (PPP), and platelet aggregation activity was tested by turbidimetry. In assay tubes, 280 μL of PRP and 10 μL each of unknown compounds with different concentrations (1,000, 500, 200, 100, and 10 μmol/L) were added, incubated for 5 min, and added with 10 μL of ADP (final concentration: 10 μmol/L) as an inducer, the maximum platelet aggregation rate within 5 min was observed and recorded, and each concentration was determined in parallel. The experimental results are shown in FIG. 9.

### Example 6 Molecular Docking Analysis of Nicotinic Acid Derivative B

The reverse docking target was collected from the following three sources: DisGeNET database (https://www.disgenet.org/, v6), Online Mendelian Inheritance in Man (OMIM) database (http://www.omim.org/, updated on Jun. 26, 2020), and GeneCards database (https://www.genecards.org/, updated on Mar. 11, 2020).

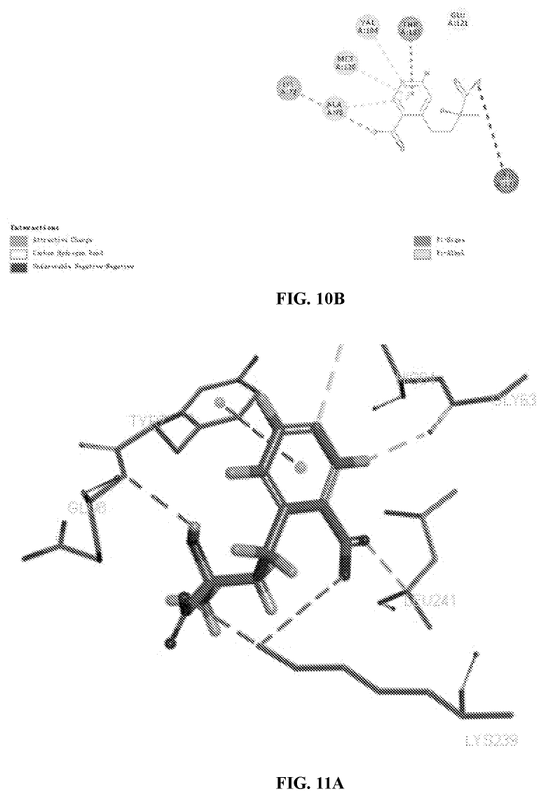

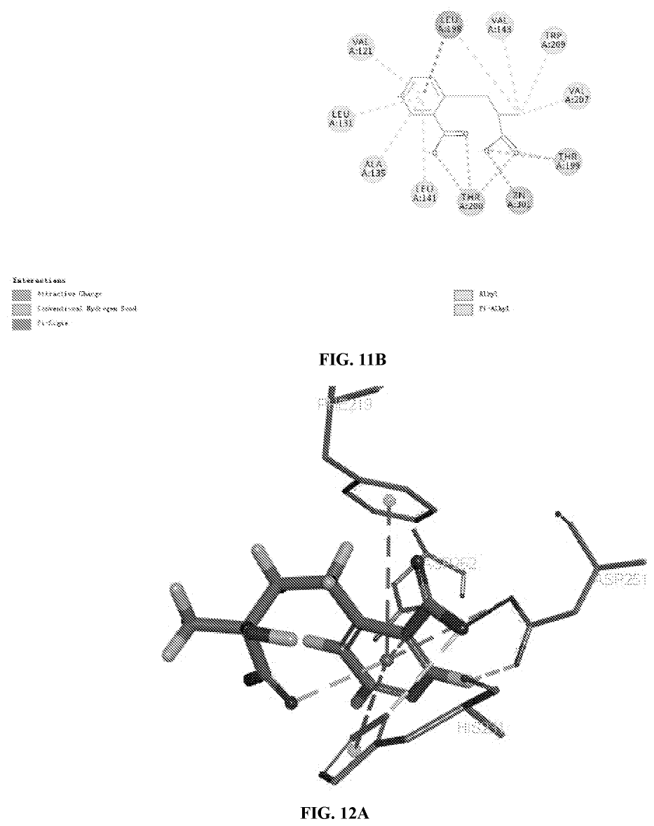

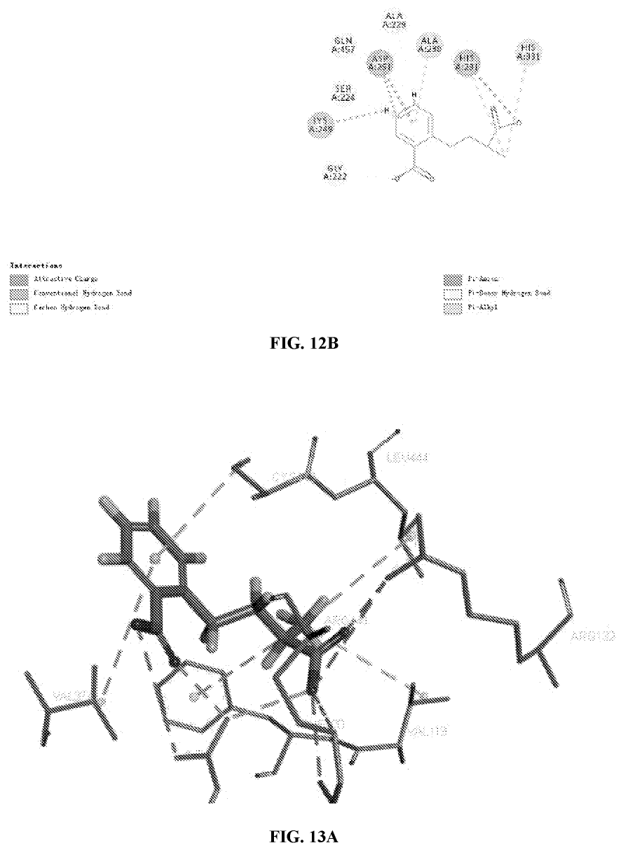

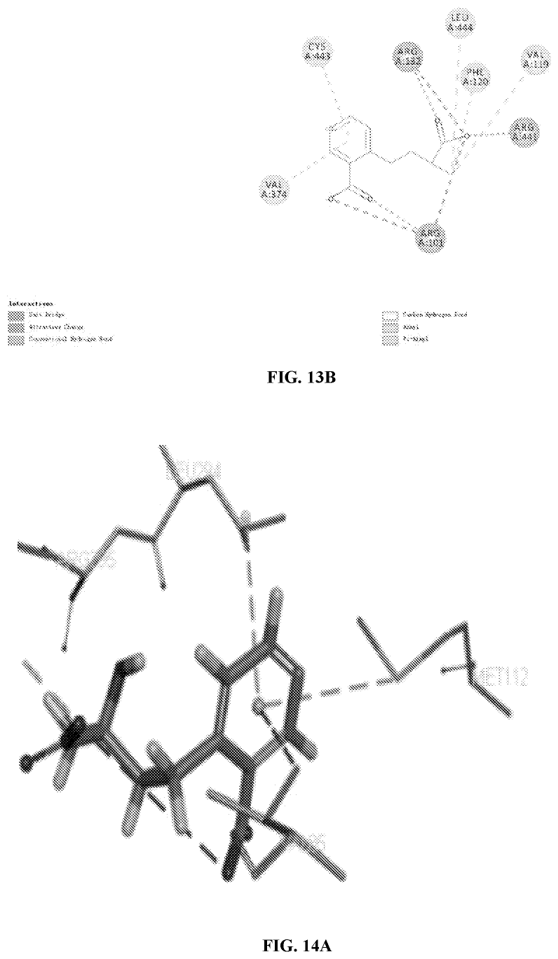

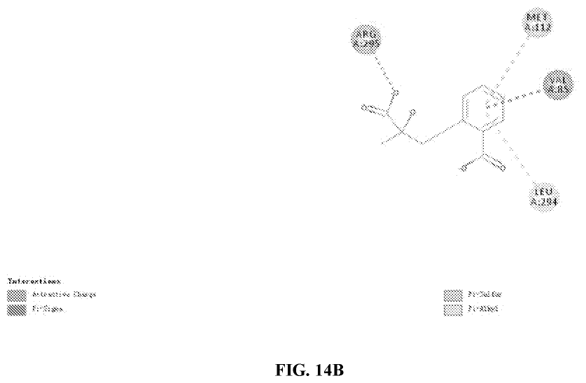

In order to verify the binding affinity of the candidate targets to the compound, simulated molecular docking was achieved using the Libdock program in Discovery Studio 16.1 (DS 16.1). As shown in Table 3, all crystal structures of the candidate targets were downloaded directly from the RCSB protein database (http://www.pdb.org/, updated on June 2020), and their resolutions were checked. In addition to co-crystallized ligands and structural water molecules, each protein was defined as a receptor, and active sites of the protein in the receptor chamber were discovered using a Discovery Studio tool; subsequently, a docking protocol was performed using Libdock to display the interactions between the components in the Discovery Studio and the differential proteins. Since Libdock could provide 10-100 predicted Libdock scores, and the positions of each binding protein in the protein binding pocket were different, the optimal Libdock score was only considered. The protein with the highest score was considered to be an assumed composite target. The experimental results are shown in FIGS. 10A-B, FIGS. 11A-B, FIGS. 12A-B, FIGS. 13A-B, and FIGS. 14A-B.

TABLE 3
Target reverse docking analysis of nicotinic acid derivative B

| Uniprot_ID | Protein | Prob | Target |
|---|---|---|---|
| P05979 | Dihydroorotate dehydrogenase (quinone) | 0.999 | 3KVJ |
| P21731 | Thromboxane A2 receptor | 0.989 | 6IIV |
| P50579 | Methionine aminopeptidase 2 | 0.915 | 5LYW |
| P30305 | Cytochrome P450 2D6 | 0.943 | 6CSD |
| Q9ULX7 | Carbonic anhydrase 14 | 0.974 | 5CJF |

1. A nicotinic acid derivative B having anti-inflammatory and immunosuppressive activity, wherein the nicotinic acid derivative B is represented by the following general molecular formula: 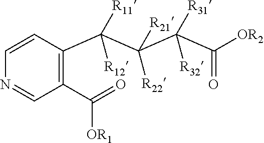
wherein R₁ and R₂ are different substitution sites on a main chain, and R₁₁′, R₂₁′, R₃₁′, R₁₂′, R₂₂′, and R₃₂′ are different substitution sites on a side chain.

2. The nicotinic acid derivative B according to claim 1, wherein the R₁ is one selected from the group consisting of H⁺, OH⁻, CH₃⁻, SH, CH₂CH₃⁻, CONH₂⁻, and NH₂⁻, and the R₂ is one selected from the group consisting of H⁺, OH⁻, CH₃⁻, SH, CH₂CH₃⁻, CONH₂⁻, and NH₂⁻.

3. The nicotinic acid derivative B according to claim 1, wherein the R₁₁′, the R₂₁′, the R₃₁′, the R₁₂′, the R₂₂′, and the R₃₂′ each are one selected from the group consisting of H⁻, OH⁻, and CH₃⁻.

4. The nicotinic acid derivative B according to claim 1, wherein the structural formula is as follows:

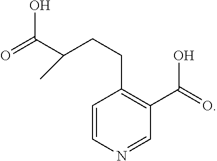 [SMILES: CC(CCc1ccncc1C(=O)O)C(=O)O]

5-9. (canceled)

10. The nicotinic acid derivative B according to claim 2, wherein the structural formula is as follows:

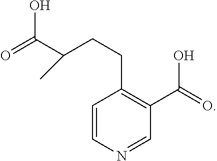 [SMILES: CC(CCc1ccncc1C(=O)O)C(=O)O]

11. The nicotinic acid derivative B according to claim 3, wherein the structural formula is as follows:

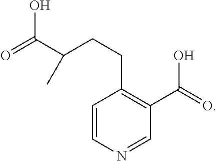 [SMILES: CC(CCc1ccncc1C(=O)O)C(=O)O]

12. The nicotinic acid derivative B according to claim 1, wherein the structural formula is as follows:

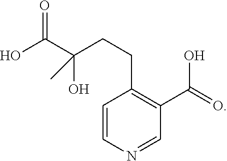 [SMILES: CC(O)(CCc1ccncc1C(=O)O)C(=O)O]

13. The nicotinic acid derivative B according to claim 2, wherein the structural formula is as follows:

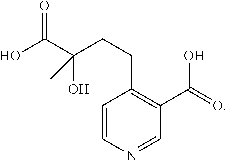 [SMILES: CC(O)(CCc1ccncc1C(=O)O)C(=O)O]

14. The nicotinic acid derivative B according to claim 3, wherein the structural formula is as follows:

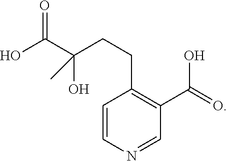 [SMILES: CC(O)(CCc1ccncc1C(=O)O)C(=O)O]

15. The nicotinic acid derivative B according to claim 1, wherein the structural formula is as follows:

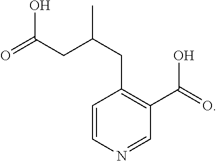 [SMILES: CC(CC(=O)O)Cc1ccncc1C(=O)O]

16. The nicotinic acid derivative B according to claim 2, wherein the structural formula is as follows:

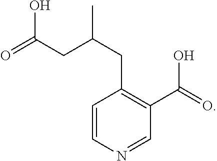 [SMILES: CC(CC(=O)O)Cc1ccncc1C(=O)O]

17. The nicotinic acid derivative B according to claim 3, wherein the structural formula is as follows:

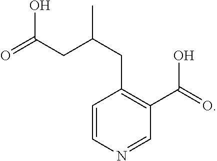 [SMILES: CC(CC(=O)O)Cc1ccncc1C(=O)O]

18. A method for treating rheumatoid arthritis, psoriasis, and other autoimmune diseases, comprising administering or applying the nicotinic acid derivative B having immunosuppressive activity according to claim 1 to a subject.

19. The method according to claim 18, wherein the R₁ is one selected from the group consisting of H⁺, OH⁻, CH₃⁻, SH, CH₂CH₃⁻, CONH₂⁻, and NH₂⁻, and the R₂ is one selected from the group consisting of H⁺, OH⁻, CH₃⁻, SH, CH₂CH₃⁻, CONH₂⁻, and NH₂⁻.

20. The method according to claim 18, wherein the R₁₁′, the R₂₁′, the R₃₁′, the R₁₂′, the R₂₂′, and the R₃₂′ each are one selected from the group consisting of H⁺, OH⁻, and CH₃⁻.

21. The method according to claim 18, wherein the structural formula is as follows:

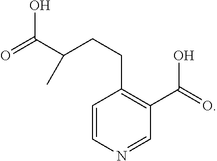 [SMILES: CC(CCc1ccncc1C(=O)O)C(=O)O]

22. An anti-inflammatory and anti-platelet aggregation medicament composition, comprising the nicotinic acid derivative B according to claim 1.

23. The composition according to claim 22, wherein the Ru is one selected from the group consisting of H⁺, OH⁻, CH₃⁻, SH, CH₂CH₃⁻, CONH₂⁻, and NH₂⁻, and the R₂ is one selected from the group consisting of H⁺, OH⁻, CH₃⁻, SH, CH₂CH₃⁻, CONH₂⁻, and NH₂⁻.

24. The composition according to claim 22, wherein the R₁₁′, the R₂₁′, the R₃₁′, the R₁₂′, the R₂₂′, and the R₃₂′ each are one selected from the group consisting of H⁺, OH⁻, and CH₃⁻.

25. The composition according to claim 22, wherein the structural formula is as follows: 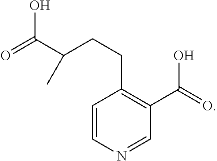 [SMILES: CC(CCc1ccncc1C(=O)O)C(=O)O]
